(19) United States
(12) Patent Application Publication
(10) Pub. No.: US 2004/0185118 A1
(43) Pub. Date: Sep. 23, 2004

(54) Health food for good sleep

(76) Inventors: Katsumichi Matsushige, Funabashi-shi (JP); Ryuichi Moriya, Tokyo (JP); Nobuki Muramatsu, Tokyo (JP)

Correspondence Address: RABIN & BERDO, P.C., Suite 500, 1101 14 Street, N.W., Washington, DC, 20005, US

(21) Appl. No.: 10/390,650

(22) Filed: Mar. 19, 2003

(51) Int. Cl. A61K 35/84; A61K 35/78; A61K 33/06

(52) U.S. Cl. 424/682; 424/195.15; 424/752; 424/728; 424/757; 424/764; 424/773; 424/774

(57) ABSTRACT

An innovative health food for good sleepy is provided for improving the factors that interfere with sleep as much as possible and is suitable for attaining good sleep and deep sleep mainly comprising herbal medicines in combination. The health food for good sleep contains a Panax ginseng extract, a Ginkgo biloba leaf extract, a Ganoderma lucidum extract, and calcium, which are mixed together in predetermined proportions. For example, the mixture contains 0.5 to 1.0 parts by weight of the Ginkgo biloba leaf extract, 0.1 to 0.5 parts by weight of the Ganoderma lucidum extract, and 0.1 to 1.0 parts by weight of calcium with respect to 1 part by weight of the Panax ginseng extract.

### BACKGROUND OF THE INVENTION

1. Field of the Invention

The present invention relates to a health food for good sleep, and in particular, the so-called health food that allows a person to have a good sleep at rest in the night by eating or drinking the health food which is not a medicine for recovering from insomnia.

2. Description of the Related Art

In recent years, for removing physical and mental fatigue, medicines, foods, health appliances, bedclothes, bath preparations, and so on have been on the market.

Medicines are originally used for disease-fighting, so that daily dietary life should be basic to maintenance of good health and prevention of a disease.

Heretofore, means for directly removing physical fatigue or the like is to take a medicine such as one known as a revitalizer or a fatigue-recovering agent.

In addition, under the present situation, symptoms related to physical fatigue, such as sleeplessness, stiff shoulder, feebleness, weakened four limbs, and so on are mainly dealt with medicines.

Furthermore, these medicines should be taken essentially under direction by the physician or the pharmacist.

In particular, with respect to sleeplessness, any sleeping drug is administered as prescribed by the physician and is only used for sick sleeplessness. On the other hand, the administration of sleeping drug for non-disease phenomena such as bad-falling sleep and restless sleep is not preferable because of being at risk of finding constitutive adverse reactions.

Most of sleeping drugs as medicines directly effect on the sleep center in the brain. Therefore, factors that interfere with sleep come to the functions of the entire body, such as pain, swelling, sick blood, excessive sensitivity to cold, decrease in the level of blood sugar, stress, nutritional balance (in particular, calcium deficiency), blood pressure abnormal, cardiac failure, anemia, gastrointestinal disturbance, urination abnormal, decrease in physical strength, and autonomic imbalance.

Many persons suffered from insomnia have two or more factors in general, so that these factors cannot be improved by taking any medicine that only effects on the sleep center in the brain.

For simultaneously improving two or more factors, two or more medicines corresponding to the respective factors should be taken. In this case, the risk of side effects may be increased.

Therefore, there is no appropriate medicine or food what is called for maintaining bodily functions to improve the body as a whole for good sleep or deep sleep.

### SUMMARY OF THE INVENTION

In consideration of the above facts, for improving the factors that interfere with sleep as much as possible, it is an object of the present invention to provide an innovative health food suitable for attaining good sleep and deep sleep mainly comprising herbal medicines (i.e., plant extracts) in combination with each other.

The foregoing object is accomplished in one embodiment by providing a health food for good sleep comprising a mixture of a Panax ginseng extract, a Ginkgo biloba leaf extract, a Ganoderma lucidum extract, and calcium at a predetermined ratio.

### BRIEF DESCRIPTION OF THE DRAWINGS

FIG. 1 is a block diagram for illustrating the process of preparing a health food for good sleep in accordance with the present invention.

### DETAILED DESCRIPTION OF PREFERRED EMBODIMENTS

Hereinafter, we will describe preferred embodiments of the present invention in detail.

A health food, which is one of the preferred embodiments of the invention, is formulated to remove the following factors, which prevent a person from having a good sleep, as much as possible. The factors include (1) body pain, (2) excessive sensitivity to cold, (3) feeling of hunger, (4) metal fatigue (accumulation of stress), (5) deficiencies of calcium and vitamins B₁, B₆, and B₁₂, (6) autonomic nervous system abnormality, (7) pressure abnormality (high or low blood pressure), (8) palpitation or irregular heartbeat, (9) anemia, (10) gastrointestinal troubles such as indigestion and disordered digestion, (11) urination troubles such as residual urine and increased urinary frequency, (12) constipation, (13) loss of bodily strength and worthlessness feeling in the entire body, and so on.

Therefore, the health food for good sleeping of the present embodiment comprises a mixture of herbal materials having positive effects of removing the above factors as much as possible and a Ginkgo biloba leaf extract. In other words, the health food of the present embodiment comprises a mixture of a Panax ginseng extract, a Ginkgo biloba leaf extract, a Ganoderma lucidum extract, and calcium at a predetermined ratio. Hereinafter, the details of each component will be described.

### (A) The Panax ginseng extract

Panax ginseng has properties of activation, mental stability, anti-aging, and so on. Panax ginseng exerts effects on various symptoms and also exerts effects on a wide range of clinical applications, so that it has been used in many Chinese medicine formulations including “ginseng soup” and “Bojun Ikki Tan”. It has been confirmed that this ginseng extract has an excellent effect on sleeplessness, so that the research results of such an effect of the ginseng extract have been reported in many documents. For instance, when the ginseng extract is given to a rat, the period of non-rapid eye movement (REM) sleep increases significantly to effect on the improvement of sleep disorder. Furthermore, it has been also confirmed that the ginseng extract has central nerve system depressing action, mental stabilizing action, cholinergic action, histamine like action, blood pressure stabilizing action, sedative action, availabilities to prevention and treatment of psychosomatic disease in autonomic imbalance, and so on.

### (B) Ginkgo Biloba Leaf Extract

The Ginkgo biloba leaf extract is already used as a medicine in other countries, particularly in Germany and France. It is always the fifth-best seller in the field of medicines, so that it is increasingly popular as a raw material for pharmaceutical production. In Japan, on the other hand, the Ginkgo biloba leaf extract is an unauthorized medicine because of being regulated under the Pharmaceutical Affairs Law, so that it is used as a health food in the present circumstances. The main component of such a Ginkgo biloba leaf extract is flavonoid. It is noted that it contains double flavone which is peculiar to the Ginkgo biloba leaf extract.

The positive effects of the Ginkgo biloba leaf extract is to normalize (protect) the capillary blood vessel in addition to control platelet agglutination, and to facilitate the flow of blood while preventing the formation of blood clot that clogs the blood vessel and leads to infarction. Therefore, the Ginkgo biloba leaf extract has a wide range of applications including arteriosclerosis, hypertension, stress syndrome, cardiac disease, cerebral apoplexy, dementia and so on.

Furthermore, in many medical documents, it has been confirmed that the Ginkgo biloba leaf extract also significantly improves sleeplessness. Therefore, the invention pays attention to this fact and thus the health food of the invention contains the Ginkgo biloba leaf extract as one of main components. Furthermore, it has been reported in medical documents that Ginkgo biloba leaf extract does not show any side effect and any contraindication when it is mixed with other food raw materials.

### (C) Ganoderma Lucidum (Reishi) Extract

The Ganoderma lucidum extract is an herbal medicine extracted from a fruiting body of Ganoderma lucidum grouped in Polyporaceae. According to the traditional Chinese medical, the Ganoderma lucidum extract has a mental stabilizing action in addition to other actions such as tonic, blood-supplying, and hydragogue actions. In addition, it shows an effect of sleep modification and mental stabilization. That is, when the Ganoderma lucidum extract is given to a rat, it is confirmed the total amount of non-REM sleep significantly increases in a dark period while not increasing in a bright period. In addition, the total amount of REM-sleep significantly increases not only in the dark period but also in the bright period. In this manner, it is confirmed that the Ganoderma lucidum extract contains a component that has a sleep-promoting effect. Therefore, in the invention, the Ganoderma lucidum extract is contained as one of main components in the health food.

### (D) Calcium

Calcium is a very important element as a main component of skeleton and tooth. It has been also medically known that the decrease in calcium content in the blood leads to an increase in abnormality of nerve excitability. The increase in nerve excitability interrupts sleep.

Therefore, in the invention, calcium is contained as one of main components in the health food for preventing the nerve from excessive excitation. The calcium may be one of various kinds of calcium materials such as pearl calcium, eggshell calcium, crushed bone calcium, oyster shell calcium, and calcium phosphate. In the invention, calcium is not limited to a specific one. Any kind of calcium may be used. However, calcium is hardly absorbed in general, compared with other elements. In this embodiment, therefore, pearl calcium is preferably used because of its excellent absorbing efficiency.

In the present embodiment, the health food is prepared by mixing 0.5 to 1.0 parts by weight of a Ginkgo biloba leaf extract, 0.1 to 0.5 parts by weight of a Ganoderma lucidum extract, and 0.1 to 1.0 parts by weight of calcium with respect to 1 part by weight of a Panax ginseng extract. Depending on consumer demands, alternatively, the mixture may be provided as dry powders, condensate with syrup or honey, granules and a tablet prepared by adding filler in the powders and coagulating the mixture in a mold, and so on. Furthermore, it may be provided as a capsulated extract, powder, or granule.

### (E) Rehmanniae Radix Stem

The Rehmanniae radix stem is provided as an extract having an effect of lowering the blood glucose level. In addition, it is also known that it shows blood-supplying, tonic, and haemostatic actions, so that it effects on diabetes, climacteric disturbance, arteriosclerosis, and so on. Furthermore, it acts for recovering from fatigue. Therefore, it becomes possible to recover from fatigue and gives a good sleep, allowing a person to fall into a deep sleep.

Next, an example of a preferable intake of each component in the health food of the present embodiment per day is as follows. On the standard scale, the Panax ginseng extract is 200 mg, the Ginkgo biloba leaf extract is 150 mg, the Ganoderma lucidum extract is 50 mg, and calcium is 100 mg. However, the present embodiment is not limited to these contents.

### (F) Tochu (Eucommia Ulmoides) Leaf

It is well known in the art that Tochu leaf is one of popular products in recent years because of being provided as a drinkable extract prepared by cutting the leaves into pieces and boiling. It has been confirmed that the Tochu leaf contains gutta-percha and has tonic, hypertensive, diuretic, muscularity-strengthening, liver-strengthening, and insomniac actions in addition to the action of lowering the sensitivity to external stimulation. From this point, the Tochu leaf may be contained as one of components in the health food of the invention.

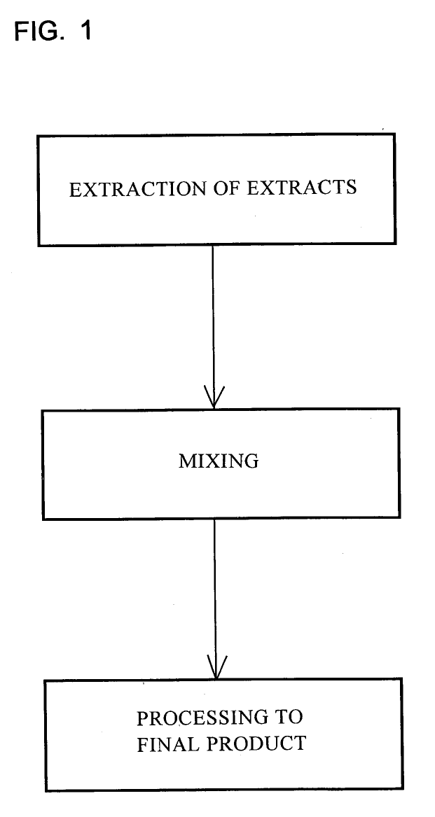

In the invention, as shown FIG. 1, each of Panax ginseng, Ginkgo biloba leaf, and Ganoderma lucidum, which are main components of the health food, is subjected to the step of extraction to prepare an extract fraction thereof. In the step of mixing, subsequently, the extracts of theses components are mixed with calcium. Alternatively, each component may be dried and provided as powders, followed by mixing together with other dried powdery components. The mixture is then mixed with calcium to obtain the same product. The step of extraction may be any extraction procedure well known in the art. The resulting mixture is further processed into the form of the final product.

### (G) Jujube (Ziziphus Jujuba)

The jujube contains sucrose, mucus, malic acid, tartaric acid, and other ingredients. The extract of Jujube has the actions of recovering from fatigue, preventing excitation of nerve to allow mental stabilization, and relieving drug effects. Thus, it is suitable for sleeplessness. From this point, the jujube extract maybe one of the components of the health food of the invention.

That is, in the case of directly providing the health food for good sleep of the invention in extract form, the mixture of extracts may be bottled. Alternatively, the mixture may be provided as a condensate by further mixing with syrup or honey, followed by being bottled. In the case of providing the mixture in powdery form, for instance, it may be provided as dried powders in a bag. In the case of providing the mixture in granulated form or tablet form, the mixture may be pulverized, followed by adding filler such as lactose in the pulverized mixture and then coagulating the mixture in a mold. Furthermore, the mixed extract, dry powders, or granules may be capsulated in a hard capsule such as a gelatin capsule or a soft capsule such as one prepared by adding glycerin as a plasticizer, water, and so on in a gelatin as a base. Therefore, the health food can be provided as a capsule type food which can be easily swallowed.

### (H) Licorice

The licorice contains glycyrrhizin. The extract of licorice has the actions of detoxication, expectorant, antitussive, pain relief, refreshing, releasing interrupted pulse, recovering from fatigue, and so on. Thus, it has the action of clearing the cause of sleeplessness.

As described above, the health food for good sleep in accordance with the present invention comprises the Panax ginseng extract, the Ginkgo biloba leaf extract, the Ganoderma lucidum (Reishi) extract, and calcium. Therefore, effects of the respective components can be acted as a synergic effect to facilitate the flow of blood while relieving each symptom which is one of the causes of sleeplessness. Therefore, it becomes possible to effectively obtain a good sleep by preventing an increase in excitability of cerebral nerve, stabilizing mental, and getting into better physical condition. Consequently, the invention provides a health food which is innovative in that herbal medicines are provided as main raw materials in combination for improving factors that prevent the consumer from having a good and deep sleep. In addition, such a health food does not exert any effect like a hypnotic. In other words, such a health food successively provides a person with a good and deep sleep at a rest time in the night when it is successively eaten or drunk to regulate the physical condition, while it does not exert any effect like a hypnotic as a conventional medicine. Therefore, the consumer would not become sleepy even immediately after eating or drinking the health food. In this case, also, the behavior of the consumer would not be relaxed.

### (I) Aurantii Nobilis Pericarpium

The Aurantii nobilis pericarpium is used as an aromatic stomachic. In addition, the extract thereof is also used for facilitating water absorption, increasing food appetite, releasing the sense of fullness, and so on. Furthermore, an offensive odor of Chinese herbal medicine can be cancelled by mixing with the Aurantii nobilis pericarpium. Therefore, it is recognized that it allows the medicine to be easily swallowed.

### (J) Chrysanthemum

The chrysanthemum is also known as a Chinese herbal medicine from a long time ago and the extract thereof has the actions of relieving headache and lightheadedness, increasing food appetite, and so on. As the chrysanthemum flower relieves headache and lightheadedness which interrupt a quiet sleep, it may be provided as one of components of the health food of the invention.

### (K) Coix Lacryma-joli (Adlay)

The adlay is used as a food or a Chinese medicine in the art from a long time ago. The extract thereof has the actions of removing swelling, pain relief, anti-inflammatory, detoxication, and so on.

In the invention, as shown in FIG. 1, each extract of Panax ginseng, Ginkgo biloba leaf, Ganoderma lucidum, Rehmanniae radix stem, Tochu leaf, Jujube, Licorice, Aurantii nobilis pericarpium, Chrysanthemum, and adlay in the step of extraction. The step of extraction may be any extraction procedure well known in the art. Each of the extracts was mixed with calcium in the step of mixing, followed by further processing into the form of the final product. Alternatively, each of the resulting components may be mixed as dry powders, followed by extracting and mixing with calcium to obtain the same product.

That is, in the case of directly providing the health food for good sleep of the invention in extract form, the mixture of extracts may be bottled. Alternatively, the mixture may be provided as a condensate by further mixing with syrup or honey, followed by being bottled. In the case of providing the mixture in powdery form, for instance, it may be provided as dried powders in a bag. In the case of providing the mixture in granulated form or tablet form, the mixture maybe pulverized, followed by adding filler such as lactose in the pulverized mixture and then coagulating the mixture in a mold. Furthermore, the mixed extract, condensate, dry powders, or granules may be capsulated in a hard capsule such as a gelatin capsule or a soft capsule such as one prepared by adding glycerin as a plasticizer, water, and so on in a gelatin as a base. Therefore, the health food can be provided as a capsule type food which can be easily swallowed.

In the present embodiment, the health food is prepared as an extract mixture by mixing 0.1 to 1.0 part by weight of a Ginkgo biloba leaf extract, 0.1 to 0.5 parts by weight of a Ganoderma lucidum extract, 0.1 to 1.0 part by weight of calcium, 0.5 to 3.0 parts by weight of a Rehmanniae radix stem extract, 0.25 to 0.75 parts by weight of a Tochu leaf extract, 1.5 to 3.5 parts by weight of a Jujube extract, 0.25 to 1.0 parts by weight of a Licorice extract, 0.1 to 0.5 parts by weight of an Aurantii nobilis pericarpium extract, 0.1 to 0.5 parts by weight of a Chrysanthemum extract, and 0.3 to 2.5 parts by weight of an adlay extract with respect to 1 part by weight of a Panax ginseng extract. Alternatively, the extract mixture may be provided as a condensate by the addition of syrup or honey. Alternatively, the extract mixture may be provided as granules or a tablet by pulverizing the mixed extract and adding a predetermined amount of lactose and the like as a filler in the powders and coagulating the mixture in a mold. Alternatively, it is also possible to capsulate the extract, liquid, powders, or granules to provide the health food in capsulate form. In the invention, furthermore, the containing ratio of each component is not limited to its upper limit described above. It may be appropriately changed if required.

Next, an example of a preferable intake of each component in the health food of the present embodiment per day is as follows. When health food for good sleep being formulated on the basis of the above mixing ratio is eaten or drunk by the consumer, it is suitable for the consumer since the intake of each of the components becomes as follows. That is, the Panax ginseng extract is 200 mg, the Ginkgo biloba leaf extract is 150 mg, the Ganoderma lucidum extract is 50 mg, the Rehmanniae radix stem extract is 400 mg, the Tochu extract was 100 mg, the Jujube extract is 550 mg, the Licorice extract was 150 mg, the Aurantii nobilis pericarpium extract is 50 mg, the Chrysanthemum extract is 50 mg, the adlay extract is 300 mg, and calcium is 100 mg. These values are standard values. Therefore, needless to say, the invention is not limited to these values.

As described above, the health food for good sleep in accordance with the invention comprises the Panax ginseng extract, the Ginkgo biloba leaf extract, the Ganoderma lucidum (Reishi) extract, calcium, the Rehmanniae radix stem extract, the Tochu (Eucommia ulmoides) leaf extract, the Jujube (Ziziphus jujuba) extract, the Licorice extract, the Aurantii nobilis pericarpium extract, the Chrysanthemum extract, and the Coix lacryma-joli (adlay) extract. Therefore, effects of the respective components can be acted as a synergic effect to facilitate the flow of blood while relieving each symptom which is one of the causes of sleeplessness. Therefore, it becomes possible to effectively obtain a good sleep or a deep sleep by preventing an increase in excitability of cerebral nerve, stabilizing mental, and getting into better physical condition.

Consequently, the invention provides a health food which is innovative in that herbal medicines are provided as main raw materials in combination for improving factors that prevent the consumer from having a good and deep sleep. In addition, such a health food does not exert any effect like a hypnotic. In other words, such a health food successively provides a person with a good and deep sleep at a rest time in the night when it is successively eaten or drunk to regulate the physical condition, while it does not exert any effect like a hypnotic as a conventional medicine. Therefore, the consumer would not become sleepy even immediately after eating or drinking the health food. In this case, also, the behavior of the consumer would not be relaxed.

What is claimed is:

1. A health food for good sleep, comprising a Panax ginseng extract, a Ginkgo biloba leaf extract, a Ganoderma lucidum extract, and calcium, which are mixed together in the proportions of:
0.5 to 1.0 part by weight of the Ginkgo biloba leaf extract;
0.1 to 0.5 parts by weight of the Ganoderma lucidum extract; and
0.1 to 1.0 part by weight of calcium,
with respect to 1 part by weight of the Panax ginseng extract.

2. A health food for good sleep as claimed in claim 1, further comprising a Rehmanniae radix stem extract, a Tochu leaf extract, a Jujube extract, a Licorice extract, an Aurantii nobilis pericarpium extract, a Chrysanthemum extract, and an adlay extract, which are mixed together in the proportions of:
0.5 to 3.0 parts by weight of the Rehmanniae radix stem extract;
0.25 to 0.75 parts by weight of the Tochu leaf extract;
1.5 to 3.5 parts by weight of the Jujube extract;
0.25 to 1.0 parts by weight of the Licorice extract;
0.1 to 0.5 parts by weight of the Aurantii nobilis pericarpium extract;
0.1 to 0.5 parts by weight of the Chrysanthemum extract; and
0.3 to 2.5 parts by weight of the adlay extract.
